(19) United States
(12) Patent Application Publication
(10) Pub. No.: US 2004/0053915 A1
(43) Pub. Date: Mar. 18, 2004

(54) Use of stimulators of soluble guanylate cyclase for treating osteoporosis

(76) Inventors: Volker Geiss, Ratingen (DE); Erich Sander, Wuppertal (DE); Johannes-Peter Stasch, Solingen (DE); Alexander Straub, Wuppertal (DE)

Correspondence Address: Jeffrey M Greenman, Bayer Corporation, 400 Morgan Lane, West Haven, CT, 06516, US

(21) Appl. No.: 10/415,708

(22) PCT Filed: Oct. 22, 2001
(86) PCT No.: PCT/EP01/12159

(30) Foreign Application Priority Data
Nov. 2, 2000 (DE) 100 54 278.6

(51) Int. Cl. A61K 31/541; A61K 31/5377; A61K 31/506

(52) U.S. Cl. 514/210.21; 514/228.5; 514/234.5; 514/252.18; 514/256

(57) ABSTRACT

The invention relates to the use of stimulators of soluble guanylate cyclase, in particular, compounds of formula (I), wherein: R1 represents a saturated or unsaturated optionally substituted C3-8 cycloalkyl or represents a saturated, unsaturated or partially unsaturated heterocyclyl, which has 3 to 8 members, contains 1-4 heteroatoms from the set N, O, S, SO, SO2, and which can be optionally substituted; R2 represents H or NH2. The invention also relates to the use of the salts, isomers and hydrates of said compounds in order to produce medicaments used for treating osteoporosis.

The present invention relates to the use of stimulators of soluble guanylate cyclase for preparing a medicament for the treatment of osteoporosis.

Osteoporosis is a systemic disorder of the skeleton which is characterized by osteolysis, i.e. a reduction in the bone mass. This results in a deterioration of the quality of the microarchitecture of the bone tissue and an increased risk of bone fracture.

There are different forms of osteoporosis. About 5% of patients are affected by secondary osteoporosis which is the result in particular of endocrine, renal and hepatic disorders (for example Cushing syndrome). Most patients (95%), however, suffer from primary osteoporosis where a distinction is made between primary idiopathic osteoporosis, type I osteoporosis and type II osteoporosis. Primary idiopathic osteoporosis affects children or young adults. Type I osteoporosis and type II osteoporosis are also referred to as postmenopausal osteoporosis and senile osteoporosis respectively. These types of osteoporosis occur in particular at a relatively high age, and the probability of an onset of the disorder increases with increasing age.

Currently, fluoride or calcium preparations (to stimulate osteoneogenesis), oestrogen, gestagen or calcitonin (to inhibit bone resorption), biphosphonates such as alendronate (to reduce bone degradation) or, at a relatively high age, even anabolics are used to treat osteoporosis.

It is thought that osteoporosis is caused by increased degradation of bone substance by osteoclasts. Osteoclasts are cells which are specialized to secrete HCl, thus degradating bone substance. Normally, in the context of the permanent restoration of bone substance in the body, bone tissue is degraded by osteoclasts and bones are regenerated by osteoblasts. Osteolytic diseases such as osteoporosis are triggered by an imbalance between bone formation and bone degradation, i.e. mainly bone degradation by osteoclasts. The bone degradation is controlled by changes in the number and activity of the osteoclasts (cf. Suda et al., J. Bone Miner. Res. 12 (1997), 869-879). There are indications that one of the most important cellular transduction systems in mammalian cells, cyclic guanosine monophosphate (cGMP), plays an important role in this context.

Together with nitrogen monoxide (NO), which is released from the endothelium and transduces hormonal and mechanical signals, cGMP forms the NO/cGMP system. The guanylate cyclases catalyse the biosynthesis of cGMP from guanosine trisposphate (GTP). The known representatives of this family can be classed into two groups both according to structural characteristics and according to the type of ligands: the particular guanylate cyclases which are stimulated by natural uretic peptides and the soluble guanylate cyclases which are stimulated by NO. The soluble guanylate cyclases consist of two subunits and, with very high probability, comprise one haem per heterodimer, which is part of the regulatory centre. This is of central importance for the mechanism of activation. NO is capable of binding to the iron atom of the haem, thus increasing the activity of the enzyme considerably. In contrast, haem-free preparations cannot be stimulated by NO. CO, too, can attack the central iron atom of the haem, but stimulation by CO is considerably lower than that by NO.

By forming cGMP and the resulting regulation of phosphodiesterases, ion channels and protein kinases, guanylate cyclase plays a decisive role in various physiological processes, in particular in the relaxation and proliferation of smooth muscle cells, platelet aggregation and platelet adhesion and neuronal signal transduction, and also in diseases which are based on a disturbance of the processes mentioned above.

It has been shown that, by inhibiting NO synthase, the activity of the osteoclasts can be enhanced considerably, resulting in increased bone degradation (Kasten et al., Proc. Nat. Acad. Sci USA 91 (1994), 3569-3573). Moreover, it has been shown that cGMP analogues which are stable to hydrolysis by phosphodiesterases (PDEs), it is possible to reduce bone degradation (Dong et al., J. Cell. Biochemistry 73 (1999), 478-487). These results indicate that cGMP is a signal which downregulates the activity of osteoclasts.

Hitherto, exclusively compounds such as organic nitrates, whose action is based on NO, have been used for therapeutic stimulation of soluble guanylate cyclase. The NO is formed by bioconversion and activates the soluble guanylate cyclase by attacking the central iron atom of the haem. In addition to the side effects, the development of tolerance is one of the decisive disadvantages of this type of treatment.

In recent years, some substances have been described which stimulate soluble guanylate cyclase directly, i.e. without prior release of NO, such as, for example, 3-(5′-hydroxymethyl-2′-furyl)-1-benzylindazole (YC-1, Wu et al., Blood 84 (1994), 4226; Mülsch et al., Br. J. Pharmacol. 120 (1997), 681), fatty acids (Goldberg et al, J. Biol. Chem. 252 (1977), 1279), diphenyliodonium hexafluorophosphate (Pettibone et al., Eur. J. Pharmacol. 116 (1985), 307), isoliquiritigenin (Yu et al., Brit. J. Pharmacol. 114 (1995), 1587) and various substituted pyrazole derivatives (WO 98/16223).

Furthermore, WO 98/16507, WO 98/23619, WO 00/06567, WO 00/06568, WO 00/06569 and WO 00/21954 describe pyrazolopyridine derivatives as stimulators of soluble guanylate cyclase.

It was an object of the present invention to find a way of treating osteoporosis.

According to the present invention, this object is achieved by using stimulators of soluble guanylate cyclase for preparing a medicament for the treatment of osteoporosis according to claim 1.

According to a preferred embodiment, the present invention relates to the use of compounds of the formula (I)

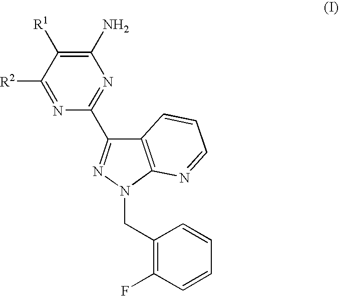

in which

R¹ represents a saturated or unsaturated, optionally substituted C₃₋₈-cycloalkyl or represents a saturated, unsaturated or partially unsaturated 3-8-membered heterocycle which may contain 1-4 heteroatoms from the group consisting of N, O, S, SO and SO₂ and which may optionally be substituted;

R² represents H or NH₂;

and salts, isomers and hydrates thereof, for preparing a medicament for the treatment of osteoporosis.

Preference according to the present invention is given to using compounds of the formula (I) in which

R¹ represents an optionally substituted cyclopropyl, cyclobutyl, cyclopentenyl, cyclopentyl, cyclohexyl, 1-hydroxycyclopropyl or 1-(fluoromethyl)cyclopropyl radical or represents optionally substituted morpholino, piperidine, piperazine, pyrrolidine, triazolyl or thiomorpholino;

R² represents H or NH₂;

and salts, isomers and hydrates thereof, for preparing a medicament for the treatment of osteoporosis.

Preference according to the present invention is given to using a compound of the formula (I) in which

R¹ represents a cyclopropyl radical,

R² represents H;

and salts, isomers and hydrates thereof, for preparing a medicament for the treatment of osteoporosis.

Preference according to the present invention is also given to using a compound of the formula (I) in which

R¹ represents morpholinyl,

R² represents NH₂;

and salts, isomers and hydrates thereof, for preparing a medicament for the treatment of osteoporosis.

The compounds of the general formula (I) according to the invention can also be present in the form of their salts. In general, salts with organic or inorganic bases or acids may be mentioned here.

In the context of the present invention, preference is given to physiologically acceptable salts. Physiologically acceptable salts of the compounds according to the invention can be salts of the substances according to the invention with mineral acids, carboxylic acids or sulphonic acids. Particular preference is given, for example, to salts with hydrochloric acid, hydrobromic acid, sulphuric acid, phosphoric acid, methanesulphonic acid, ethanesulphonic acid, p-toluenesulphonic acid, benzenesulphonic acid, naphthalenedisulphonic acid, acetic acid, propionic acid, lactic acid, tartaric acid, citric acid, fumaric acid, maleic acid or benzoic acid.

Physiologically acceptable salts can also be metal or ammonium salts of the compounds according to the invention which have a free carboxyl group. Particular preference is given, for example, to sodium salts, potassium salts, magnesium salts or calcium salts, and also to ammonium salts derived from ammonia or organic amines, such as, for example, ethylamine, di- or triethylamine, di- or triethanolamine, dicyclohexylamine, dimethylaminoethanol, arginine, lysine or ethylenediamine.

The compounds according to the invention can exist in stereoisomeric forms which are either like image and mirror image (enantiomers) or which are not like image and mirror image (diastereomers). The invention relates both to the enantiomers or diastereomers and to their respective mixtures. The racemic forms, like the diastereomers, can be separated in the known manner, for example by chromatographic separation, into the stereoisomerically uniform components. Double bonds present in the compounds according to the invention can be in the cis or trans configuration (Z or E form).

Furthermore, certain compounds can be present in tautomeric forms. This is known to the person skilled in the art, and such compounds are likewise included in the scope of the invention.

Furthermore, the compounds according to the invention can be present in the form of their hydrates, the number of water molecules attached to the molecule depending on the particular compound according to the invention.

In the context of the present invention, the substituents are, unless indicated otherwise, generally as defined below:

Cycloalkyl generally represents a cyclic hydrocarbon radical having 3 to 8 carbon atoms. Cyclopropyl, cyclopentyl and cyclohexyl are preferred. Examples which may be mentioned are cyclopropyl, cyclopentyl, cyclohexyl, cycloheptyl and cyclooctyl.

In the context of the invention, heterocycle generally represents a saturated, unsaturated or aromatic 3- to 8-membered, for example 5- or 6-membered, heterocycle which may contain up to 4 heteroatoms selected from the group consisting of S, N and O and which, in the case of a nitrogen atom, may also be attached via the latter. Examples which may be mentioned are: oxadiazolyl, thiadiazolyl, pyrazolyl, pyridyl, pyrimidinyl, pyridazinyl, pyrazinyl, thienyl, furyl, pyrrolyl, pyrrolidinyl, piperazinyl, tetra-hydropyranyl, tetrahydrofuranyl, 1,2,3 triazolyl, thiazolyl, oxazolyl, imidazolyl, morpholinyl or piperidyl. Preference is given to thiazolyl, furyl, oxazolyl, pyrazolyl, triazolyl, pyridyl, pyrimidinyl, pyridazinyl and tetrahydropyranyl. The term ‘heteroaryl’ (or ‘hetaryl’) denotes an aromatic heterocyclic radical.

The synthesis of the compounds of the formula (I) according to the invention is described in the Laid-Open Specifications WO 00/06568 and WO 00/6569, the relevant content of which is expressly incorporated herein by way of reference.

In addition, the invention includes the use of a combination of stimulators of soluble guanylate cyclase, in particular of the compounds of the general formula (I) according to the invention, with organic nitrates and NO donors for preparing a medicament for the treatment of osteoporosis.

In the context of the invention, organic nitrates and NO donors are, in general, substances which unfold their therapeutic action by releasing NO or NO species. Preference is given to sodium nitroprusside, glycerol trinitrate, isosorbide dinitrate, isosorbide mononitrate, molsidomine and SIN-1.

Moreover, the invention includes the use of a combination of stimulators of soluble guanylate cyclase, in particular of the compounds of the general formula (I) according to the invention, with compounds which inhibit the degradation of cyclic guanosine monophosphate (cGMP), for preparing a medicament for the treatment of osteoporosis. These compounds which inhibit the degradation of cyclic guanosine monophosphate (cGMP) are in particular inhibitors of the phosphodiesterases of 1, 2 and 5; nomenclature according to Beavo and Reifsnyder (1990) TiPS 11 pp. 150-155. These inhibitors potentiate the effect of the compound according to the invention and increase the desired pharmacological effect.

According to the present invention, the stimulators of soluble guanylate cyclase can be administered in pharmaceutical preparations which, in addition to the stimulators of soluble guanylate cyclase, in particular the compounds of the general formula (I) according to the invention, contain non-toxic, inert pharmaceutically acceptable excipients.

The active compounds, if appropriate in one or more of the abovementioned excipients, can also be present in microencapsulated form.

In the abovementioned pharmaceutical preparations, the therapeutically active compounds, in particular the compounds of the general formula (I), should be present in a concentration of from about 0.1 to 99.5, preferably from about 0.5 to 95, % by weight of the total mixture.

In addition to stimulators of soluble guanylate cyclase, in particular the compounds of the general formula (I) according to the invention, the abovementioned pharmaceutical preparations may also comprise further pharmaceutically active compounds.

In general, it has been found to be advantageous, both in human and veterinary medicine, to administer the active compound(s) according to the invention in total amounts of from about 0.01 to about 700, preferably from 0.01 to 100, mg/kg of body weight per 24 hours, if appropriate in the form of a plurality of individual doses, to obtain the desired results. An individual dose comprises the active compound(s) according to the invention preferably in amounts of from about 0.1 to about 80, in particular from 0.1 to 30, mg/kg of body weight.

Demonstration of Increased Bone Formation Following Addition of Stimulators of Soluble Guanylate Cyclase

For a period of 4 weeks, one of the two compounds listed in Table 1 was administered, in each case in one of the concentrations given in Table 1, once per day via a stomach tube to 10 male and 10 female rats of the strain HsdCpb:Wu. When compound 1 was administered in a concentration of 50 mg/kg or more or compound 2 was administered in a concentration of 5 mg/kg or more, all test animals showed increased bone formation in the area of the spongioid bone proximally and distally to the epiphyseal arcus of the great tubular bones after the test period. Increased bone formation was characterized by a widening of the bone trabeculae while the physiological spongioid structure was preserved. Pathological endosteal or periosteal changes were not noticed.

TABLE 1

| | | Concentrations |
|---|---|---|
| Compound | Structure | Administered |
| 1 | 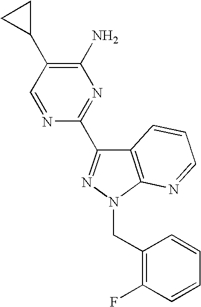 | 1 mg/kg 5 mg/kg 25 mg/kg |
| 2 | 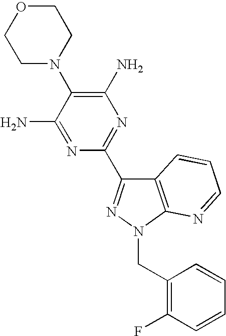 | 5 mg/kg 15 mg/kg 50 mg/kg |

1. Use of stimulators of soluble guanylate cyclase for preparing a medicament for the treatment of osteoporosis.

2. Use of compound of the formula (I) 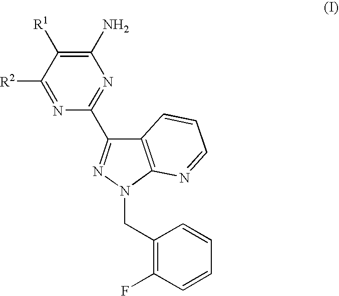
in which
R¹ represents a saturated or unsaturated, optionally substituted C₃₋₈-cycloalkyl or represents a saturated, unsaturated or partially unsaturated 3-8-membered heterocycle which may contain 1-4 heteroatoms from the group consisting of N, O, S, SO and SO₂ and which may optionally be substituted;
R² represents H or NH₂;
and salts, isomers and hydrates thereof, for preparing a medicament for the treatment of osteoporosis.

3. Use according to claim 2,
in which
R¹ represents an optionally substituted cyclopropyl, cyclobutyl, cyclopentenyl, cyclopentyl, cyclohexyl, 1-hydroxycyclopropyl or 1-(fluoromethyl)cyclopropyl radical or represents optionally substituted morpholinyl, piperidinyl, piperazinyl, pyrrolidinyl, triazolyl or thiomorpholinyl;
R² represents H or NH₂;
and salts, isomers and hydrates thereof, for preparing a medicament for the treatment of osteoporosis.

4. Use according to claim 2,
in which
R¹ represents a cyclopropyl radical,
R² represents H;
and salts, isomers and hydrates thereof, for preparing a medicament for the treatment of osteoporosis.

5. Use according to claim 2,
in which
R¹ represents morpholinyl,
R² represents NH₂;
and salts, isomers and hydrates thereof, for preparing a medicament for the treatment of osteoporosis.

6. Use according to any of the preceding claims, where the compounds set forth in any of the preceding claims are used in combination with organic nitrates or NO donors.

7. Use according to any of the preceding claims, where the compounds set forth in any of the preceding claims are used in combination with compounds which inhibit the degradation of cyclic guanosine monophosphate (cGMP).
